(19) United States
(12) Patent Application Publication
(10) Pub. No.: US 2006/0247180 A1
(43) Pub. Date: Nov. 2, 2006

(54) Purgative composition and uses thereof

(76) Inventors: James L. Bergey, Chester Springs, PA (US); Martin Rose, Bethesda, MD (US)

Correspondence Address: Min, Hsieh & Hack, LLP;c/o PortfolioIP, P.O. Box 52050, Minneapolis, MN, 55402, US

(21) Appl. No.: 11/117,436

(22) Filed: Apr. 29, 2005

(51) Int. Cl. A61K 31/7012; A61K 31/70; A61K 31/715

(52) U.S. Cl. 514/23; 514/53; 514/61

(57) ABSTRACT

A purgative composition containing a purgative effective amount of a halogenated carbohydrate is disclosed.

### DESCRIPTION OF THE DISCLOSURE

1. Field of the Disclosure

The present disclosure relates to a purgative composition, a laxative composition, and to methods of making and using them.

2. Background of the Disclosure

In certain medical procedures, for example, colonoscopies, radiographic examinations, and bowel and gynecologic surgery, it is often critical that the colon be emptied as completely as possible. For example, in order to obtain satisfactory radiographs it is often essential that the intestines be cleansed sufficiently, particularly with regard to the elimination of gas from the colon. The same condition also applies when the colon is preoperatively prepared for surgery, or for diagnostic procedures such as colonoscopies, in which case it is also necessary to remove fecal waste materials.

Known colonic purgative procedures involve emptying of the colon using enemas, such as large volume water enemas, hypertonic aqueous solution enemas, and enemas containing laxative agents. It has, however, been recognized that the use of enemas may be injurious to the patient. The problems with enemas, aside from the often problematic methods of enema administration, include incomplete evacuation of the bowels, the need for repeat administrations, and the inclusion of certain chemicals that may have an irritating effect on the colonic walls. Furthermore, because it is often necessary to employ repeated washout enemas to clear the colon effectively, the potential for such chemical irritation can be greatly increased.

More recently, a number of orally administered liquid pharmaceutical compositions have been developed for use as gastrointestinal washes for diagnostic purposes or for use as purgatives. Such preparations comprise aqueous solutions of polyethylene glycol and electrolytes such as sodium sulfate, sodium bicarbonate, sodium chloride and potassium chloride. These orally administered compositions can be useful in the rapid washing of the colon for diagnostic purposes. Commercially available products embodying these formulations typically utilize a polyethylene glycol formula serving as a non-absorbable osmotic agent with a mixture of electrolytes for replenishment, so that patients do not become dehydrated. Patients are required to ingest a significant amount of volume for purgation which may include one eight ounce glass every ten minutes for up to four liters of fluid. Due to the fact that the volume is so high, use of this type of formulation is frequently associated with distention and nausea on a significant scale.

Another serious drawback of these known preparations is their unpleasant, bitter, saline taste which in the more sensitive patients can lead to vomiting—thereby preventing ingestion. However, as the requirement of solution isotonicity is necessary to obtain the aforesaid advantages, the introduction of water soluble adjuvants, for example, to alter taste, should be avoided.

Moreover, it is well recognized that the addition of appreciable quantities of substances which can be fermented by the intestinal flora should be avoided. This is because flammable gas could form which could be dangerous in the case of a colonoscopy with electrocautery.

Another alternative is an aqueous solution or a solid dosage form of phosphate salts. The aqueous phosphate salt solution produces a tremendous osmotic effect on the intra-luminal contents of the bowel and therefore, evacuation of the bowel occurs with a tremendous increase in the influx of water and electrolytes into the colon. A drawback of the concentrated aqueous phosphate solution administration is that the aqueous solution is extremely unpalatable, so much so that the recommended dosage form is administered ice cold so as to minimize the objectionable saline taste. Patients often complain of severe nausea and vomiting, secondary to the extremely salty taste of the preparation. Frequently, patients cannot tolerate the ingestion of this preparation at the initial dose, and often the second dose becomes even more problematic due to the unpalatable extremely salty taste. This is the case even when the taste is partially masked by the use of flavoring agents. Thus, while concentrated purgation solutions represent a slight improvement over other methods of inducing purgation, the short comings of these solutions are readily apparent.

A need exists for a purgative and/or laxative composition that can be pleasant tasting and can sufficiently cleanse the intestines of gas and fecal matter.

### SUMMARY OF THE DISCLOSURE

In various aspects of this disclosure, there is disclosed a purgative composition comprising a purgative effective amount of a halogenated carbohydrate.

In various aspects of this disclosure, there is disclosed a laxative composition comprising a laxative effective amount of a halogenated carbohydrate.

In various aspects of this disclosure, there is disclosed a method for inducing purgation in a patient comprising administering to a patient a purgative composition comprising a purgative effective amount of a halogenated carbohydrate.

In various aspects of this disclosure, there is disclosed a method for inducing laxation in a patient comprising administering to a patient a laxative composition comprising a laxative effective amount of a halogenated carbohydrate.

Additional objects and advantages of the disclosure will be set forth in part in the description which follows, and in part will be obvious from the description, or may be learned by practice of the disclosure. The objects and advantages of the disclosure will be realized and attained by means of the elements and combinations particularly pointed out in the appended claims.

It is to be understood that both the foregoing general description and the following detailed description are exemplary and explanatory only and are not restrictive of the disclosure, as claimed.

### DESCRIPTION OF THE EMBODIMENTS

The present disclosure relates to a purgative composition comprising a purgative effective amount of a halogenated carbohydrate.

The disclosed composition can comprise a halogenated carbohydrate selected from the group consisting of a halogenated monosaccharide, halogenated polysaccharide, halogenated oligosaccharide, halogenated disaccharide, and halogenated trisaccharide. Any halogenated carbohydrate can be used so long as the carbohydrate does not ferment. One of ordinary skill in the art can understand that fermentation of a halogenated carbohydrate can result in the formation of gases which could explode. Moreover, the halogenated carbohydrate should be poorly absorbed by the intestine.

In an embodiment, the halogenated carbohydrate can be represented by formula (I): 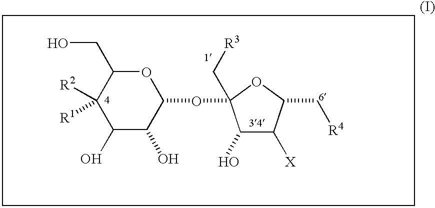
wherein X can be a halogen atom; R¹ and R², can be identical or different, and can be selected from the group consisting of a hydroxy group, a hydrogen atom, and a halogen atom; R³ and R⁴ can be identical or different and can be a halogen atom or a hydroxy group; wherein at least one of R¹, R², and R³ can be a halogen atom; with the proviso that when R², R³, and R⁴ are all chlorine atoms, X is not a chlorine atom. One of ordinary skill in the art would know a halogen atom, for example, one of the group VIIA elements, such as fluorine, chlorine, bromine, iodine, and astatine. Methods of making a halogenated carbohydrate are known to one of ordinary skill in the art, as disclosed in EP 0 073093, the disclosure of which is hereby incorporated by reference.

In another embodiment, the halogenated carbohydrate can be sucralose. Sucralose (4,1′,6′-trichloro-4,1′,6′-trideoxy-galactosucrose), a high intensity sweetener made from sucrose, can be used in many food and beverage applications because it is nontoxic and has been shown to be non-carcinogenic. See Young, D. A., et al., “The Influence of sucralose on bacterial metabolism,” J. Dent. Res., 69(8):1480-4 (1990). Sucralose, unlike many artificial sweeteners, can be used in cooking and baking with no loss of sweetening power.

Sucralose is generally made following the procedures set forth in U.S. Pat. Nos. 4,362,869; 4,380,476; 4,801,700; 4,950,746; 5,470,969; 5,498,709; 6,646,121; and 6,809,198. In all these procedures, one of the final steps in the synthesis is a deacylation followed by the crystallization of the sucralose. Laboratory scale methods for crystallizing sucralose have been described in U.S. Pat. Nos. 4,343,934; 5,141,860; 4,977,254; 4,783,526; 4,380,476; 5,298,611; 4,362,869; 4,801,700; and 4,980,463.

The term “purgative effective amount” or “purgative effective dosage” is used throughout the specification to describe the amount or concentration of a halogenated carbohydrate used herein that may be effective for producing a purgative effect, i.e., the elimination or evacuation from the intestines of their contents. In an embodiment, the composition may comprise a purgative effective amount of a halogenated carbohydrate comprising from about 1.33×10⁻³ to about 2.67×10⁻² mols/kg, for example from about 1.45×10⁻³ to about 2.00×10⁻² mols/kg, and as a further example from about 1.50×10⁻³ to about 1.00×10⁻² mols/kg.

The term “patient” is used throughout the specification to describe an animal, for example a human, to whom treatment with a disclosed composition may be provided. For treatment of those conditions that are specific for a specific animal such as a human patient, the term patient refers to that specific animal. In most instances in the disclosure, the term “patient” will refer to human patients.

The physiology of intestinal secretion and absorption is generally well known as reflected in the reported literature. While not being limited to any particular theory, a halogenated carbohydrate, such as sucralose, can be a water-soluble molecule that is poorly absorbed by the intestine. This can create an increase in intra-luminal fluid of the small bowel to a significant degree and/or creating favorable osmotic conditions in the intestine which allows for a net secretion of sodium and water into the lumen. In addition, the osmotic effect of the halogenated carbohydrate can create a purgative and/or laxative effect. This can allow for tremendous fluxes of water to be present within the gastrointestinal lumen which exhibits increased motility, thus producing a purgative and/or a laxative effect.

For example, upon ingestion a halogenated carbohydrate, like phosphate salts, can cause a tremendous amount of water to be drawn into the intestine. This influx of water can cause an increase in intraluminal pressure, which in turn can exert a mechanical stimulus causing an increase in intestinal motility.

In an embodiment, the composition can be laxative composition and can comprise a laxative effective amount of a halogenated carbohydrate. The term “laxative effective amount” or “laxative effective dosage” is used throughout the specification to describe the amount or concentration of a halogenated carbohydrate used herein which can be effective for producing a laxative effect, i.e., a mild loosening of the bowels, such as an elimination of a soft, formed stool. In an embodiment, the composition may comprise a laxative effective amount of a halogenated carbohydrate comprising from about 6.65×10⁻⁵ mol/kg to about 1.33×10⁻², for example from about 1.00×10⁻⁴ to about 1.00×10⁻², and as a further example from about 1.00×10⁻³ to about 1.00×10⁻¹ mol/kg.

The disclosed composition can be formed into an easily administered oral dosage form selected from the group consisting of a solid dosage form, such as a tablet, capsule, or powder and an aqueous dosage form for delivery to a patient. In an embodiment, the powder can be added to an aqueous solution, such as water. In another embodiment, the powder can be added, such as sprinkled, onto or into an ingestible product, such as a foodstuff.

Methods of making solid dosage forms are well known in the art, such as admixing components in a ribbon blender or other similar mung apparatus to effect complete mixing of the components. For example, when forming tablets comprising the purgative composition, it will be appreciated that the halogenated carbohydrate can be compressed into a uniform mixture and can optionally include at least one excipient.

The disclosed composition can further include at least one excipient selected from the group consisting of purgative salts, binding agents, dispersal agents, buffering agents, sweetening agents, debittering agents, flavoring agents, pH stabilizers, acidifying agents, preservatives, desweetening agents, and coloring agents. The at least one excipient can exhibit an osmotic effect. Moreover, the at least one excipient can be highly water soluble. In an embodiment wherein the excipient is not water soluble, then it should be used in an amount that would not impede any diagnostic procedures, such as those that would require visualization of the colon. For example, the at least one excipient can be present in the composition in any desired or effective amount so long as they do not interfere with the purgative and/or laxative effect of the halogenated carbohydrate. In an embodiment, the excipient can be present in an amount ranging from about 0.025% to about 25% by weight, and for example from about 1% to about 20% by weight. One of ordinary skill can readily modify the excipient combined with the halogenated carbohydrate in order to optimize the composition for oral delivery.

The term “salt” or “purgative salt” is used throughout the present application to describe at least one anhydrous compound which can find use in the disclosed composition. Salts can be found in their anhydrous form or in a hydrated crystalline form (i.e., complexed or crystallized with at least one molecule of water). Purgative salts for use herein include, but are not limited to, Mg₃(PO₄)₂, MgHPO₄, Mg(H₂ PO₄)₂, MgSO₄, MgCl₂, Na₂SO₄, sodium tartrate, potassium tartrate, magnesium tartrate, and mixtures thereof. The magnesium phosphate salts can be used in an embodiment because of the dual effect which can be produced by both the phosphate anion and the magnesium cation. It is believed that as a result of this dual action, the magnesium phosphate salts can be utilized in amounts which can be considered “low dose”, i.e. in an amount which is unexpectedly low based upon or compared to other salts, such as sodium phosphate salts which find use in anhydrous purgative compositions.

The term “anhydrous” is used throughout the specification to describe the form in which the purgative salts can be administered. Anhydrous formulations can be those which exclude water from the formulations, except, in such instances where the salt is hydrated or otherwise complexed with small amounts of water.

The binding agent for use in the disclosed composition can be a pharmaceutically acceptable binder and can be one which produces no appreciable osmotic effects. Examples of useful binding agents include, but are not limited to, non-ionic detergents from the Pluronic™ series, such as Pluronic F-68 (a trademark of BASF-Wyandotte Chemicals, defined as a condensate of ethylene oxide with a condensate of propylene oxide and propylene glycol), related non-ionic surfactants, and mechanical adhesives such as polyvinyl alcohol and sodium carboxymethylcellulose, among numerous others. Microcrystalline cellulose (MCC) can also be used to enhance the compactability of the halogenated carbohydrate into a solid dosage form.

The composition can also include dispersal agents which will facilitate dissolution of the solid dosage form contents in the gastrointestine of the patient. For example, the dispersal agent can be a pharmaceutically acceptable dispersal agent and can be one which also produces no appreciable osmotic effects. Examples of acceptable dispersal agents include, but are not limited to, microcrystalline cellulose (which is also useful as a compacting agent) and anhydrous lactose. In an embodiment, AC-DI-SOL, a cross-linked starch can be used as the dispersal agent.

In another embodiment, the composition can also include a buffering agent to minimize any acid imbalance which may accompany ingestion of the purgative composition. Suitable buffering agents include, but are not limited to, magnesium hydroxide, aluminum hydroxide, calcium carbonate, magnesium carbonate, and the like.

The disclosed composition can also include an additional sweetening agent, other than the halogenated carbohydrate. Suitable sweetening agents include, but are not limited to, sugar sweeteners such as monosaccharides, disaccharides and polysaccharides. Examples of suitable sugar sweeteners include, but are not limited to, xylose, ribose, glucose, mannose, galactose, fructose, dextrose, sucrose, maltose, partially hydrolyzed starch or corn syrup solids and sugar alcohols such as sorbitol, xylitol, mannitol, glycerin, and combinations thereof. In an embodiment, the type of glycerin used can be U.S.P. grade. The amount of sugar sweetener used in the composition can vary depending on the degree of sweetening desired for the particular composition. The sweetening agent, such as sugar sweeteners and artificial sweeteners, should be used in minute amounts in order to prevent the buildup of gas in the intestine.

Artificial sweeteners can be employed in place of or in addition to sugar sweeteners as the sweetening agent. For example, the composition can comprise an artificial sweetener as the sweetening agent including, but not limited to, aspartame, cyclamates, saccharin, acesulfame K, and mixtures thereof. In an embodiment, the artificial sweetener is not sucralose. The amount of artificial sweetener used in the composition can vary depending on the degree of sweetening desired for the particular composition.

In another embodiment, a debittering agent can be employed. Debittering agents include, but are not limited to, natural debittering agents, artificial debittering agents or debittering agents that can inhibit a chemosensory response in the mouth or nose, and mixtures thereof. Debittering agents for use herein can be commercially available, such as those marketed under the names Prosweet FL N&A K (by Virginia Dare), Bitterness Modifier 36734 (by Bush, Boake and Allen, Inc.), Natural Taste Masker 501.441/A and Special Taste Masker Compound 501.437/A (by Firmenich, Inc.), and can be identified by those skilled in the art.

Optional flavoring agents added to the composition should be of the type and amount desired for the particular suspension to meet the preferences dictated by the intended consumer of such suspension such as an adult or pediatric patient. Suitable flavoring agents include, but are not limited to, natural flavors, natural fruit flavors, artificial flavors, artificial fruit flavors, flavor enhancers, and mixtures thereof. Natural flavors, artificial flavors, and mixtures thereof include, but are not limited to, mint (such as peppermint or spearmint), menthol, cinnamon, vanilla, artificial vanilla, chocolate, artificial chocolate, and bubblegum. Natural fruit flavors, artificial fruit flavors, and mixtures thereof include, but are not limited to, cherry, grape, orange, strawberry, and lemon. Flavor enhancers include, but are not limited to, citric acid. Flavoring agents can be present as a minor component (such as less than 50% by weight) of the composition in amounts effective to provide a palatable flavor to the composition.

The composition can also comprise pH stabilizers such as citric acid. The pH stabilizers can stabilize the pH of the composition and may prevent microbial growth.

It may also be desirable to include an acidifying agent to the disclosed composition. The acidifying agents that may be applicable for use herein are those which can be acidic in a liquid vehicle and can be capable of lowering and maintaining the pH of the liquid vehicle below about pH 5. Non-limiting examples of an acidifying agent include citric acid, sodium ascorbate, and ascorbic acid. In the case of the present composition, citric acid can be used.

Preservatives useful in the disclosed composition include, but are not limited to, sodium benzoate, potassium sorbate, salts of edetate (also known as salts of ethylenediaminetetraacetic acid, or EDTA, such as disodium edetate), parabens (such as methyl, ethyl, propyl and butyl p-hydroxybenzoic acids esters or mixtures thereof), and mixtures thereof. The preservatives listed above can be exemplary, but each preservative must be evaluated on an empirical basis, in each composition, to assure the compatibility and efficacy of the preservative. Methods for evaluating the efficacy of preservatives in pharmaceutical compositions are known to those skilled in the art. Sodium benzoate, propylparaben, butylparaben, and mixtures thereof can be used in an embodiment.

A coloring agent also can be incorporated in the composition. The coloring agents should be selected to avoid chemical incompatibilities with the other ingredients in the composition. Suitable coloring agents for use in a pharmaceutical composition are well known to those skilled in the art.

Suitable desweetening agents include those compounds disclosed in United Kingdom Patent Application 2,157,148, and U.S. Pat. No. 4,567,053, the disclosures of both of which are hereby incorporated by reference, such as compounds which can be ethers or thioethers of acetic acid derivatives. Other suitable desweetening agents include the salts of substituted benzoylalkyl carboxylic acids disclosed in U.S. Pat. No. 4,544,565, which patent is incorporated hereby by reference; 3-aminobenzenesulfonic acid and derivatives thereof disclosed in U.S. Pat. No. 4,642,240, which patent is incorporated hereby by reference; the substituted phenylalkyl carboxylic acid salts and substituted phenyl ketoalkyl carboxylic acid salts disclosed in U.S. Pat. No. 4,567,053, which patent is incorporated hereby by reference; and the substituted benzoyloxy acetic and 2-propionic acid salt derivatives disclosed in United Kingdom Patent Application 2,180,534, which application is incorporated herein by reference. In an embodiment, the desweetening agent can be selected from the group consisting of zinc sulfate, 2-p-methoxyphenoxypropionic acid manufactured under the trademark LACTISOLE™ by Tate & Lyle, p-methoxybenzylacetic acid, and mixtures thereof.

In an embodiment, there is disclosed a method for inducing purgation in a patient comprising administering to a patient a purgative composition comprising a purgative effective amount of a halogenated carbohydrate. In another embodiment, there is disclosed a method for inducing laxation in a patient comprising administering to a patient a laxative composition comprising a laxative effective amount of halogenated carbohydrate.

Other embodiments of the disclosure will be apparent to those skilled in the art from consideration of the specification and practice of the disclosure. It is intended that the specification and examples be considered as exemplary only, with a true scope and spirit of the invention being indicated by the following claims.

What is claimed is:

1. A purgative composition comprising a purgative effective amount of a halogenated carbohydrate.

2. The composition of claim 1, where the composition is in the form of a solid dosage.

3. The composition of claim 2, wherein the solid dosage is selected from the group consisting of a tablet, a capsule, and a powder.

4. The composition of claim 1, wherein the halogenated carbohydrate is selected from the group consisting of a halogenated monosaccharide, halogenated polysaccharide, halogenated oligosaccharide, halogenated disaccharide, and halogenated trisaccharide.

5. The composition of claim 1, wherein the halogenated carbohydrate is sucralose.

6. The composition of claim 1, wherein the purgative effective amount of the halogenated carbohydrate is in a range from about 1.33×10⁻³ to about 2.67×10⁻² mols/kg.

7. The composition of claim 1, wherein the purgative effective amount of the halogenated carbohydrate is in a range from about 1.45×10⁻³ to about 2.00×10⁻² mols/kg.

8. The composition of claim 1, further comprising at least one excipient selected from the group consisting of a purgative salts, binding agents, dispersal agents, buffering agents, sweetening agents, debittering agents, flavoring agents, pH stabilizers, acidifying agents, preservatives, desweetening agents, and coloring agents.

9. A laxative composition comprising a laxative effective amount of a halogenated carbohydrate.

10. The composition of claim 9, where the composition is in the form of a solid dosage.

11. The composition of claim 10, wherein the solid dosage is selected from the group consisting of a tablet, a capsule, and a powder.

12. The composition of claim 9, wherein the halogenated carbohydrate is selected from the group consisting of a halogenated monosaccharide, halogenated polysaccharide, halogenated oligosaccharide, halogenated disaccharide, and halogenated trisaccharide.

13. The composition of claim 9, wherein the halogenated carbohydrate is sucralose.

14. The composition of claim 9, wherein the laxative effective amount of the halogenated carbohydrate is in a range from about 6.65×10⁻⁵ mol/kg to about 1.33×10⁻² mol/kg.

15. The composition of claim 9, wherein the laxative effective amount of the halogenated carbohydrate is in a range from about 1.00×10⁻⁴ to about 1.00×10⁻² mol/kg.

16. The composition of claim 9, further comprising at least one excipient selected from the group consisting of a purgative salts, binding agents, dispersal agents, buffering agents, sweetening agents, debittering agents, flavoring agents, pH stabilizers, acidifying agents, preservatives, desweetening agents, and coloring agents.

17. A method of inducing purgation in a patient comprising administering to a patient a purgative composition comprising a purgative effective amount of a halogenated carbohydrate.

18. The method of claim 17, wherein the purgative effective amount of the halogenated carbohydrate is in a range from about 1.33×10⁻³ to about 2.67×10⁻² mols/kg.

19. A method of inducing laxation in a patient comprising administering to a patient a laxative composition comprising a laxative effective amount of a halogenated carbohydrate.

20. The method of claim 19, wherein the laxative effective amount of halogenated carbohydrate is in a range from about 6.65×10⁻⁵ mol/kg to about 1.33×10⁻² mol/kg.
